(19) United States
(12) Patent Application Publication
(10) Pub. No.: US 2007/0042340 A1
(43) Pub. Date: Feb. 22, 2007

(54) Method and apparatus for protecting biological specimens

(76) Inventors: Juha Kononen, Jyvaskyla (FI); Dan Rohwer-Nutter, Sun Prairie, WI (US)

Correspondence Address: FISH & RICHARDSON P.C., P.O. BOX 1022, MINNEAPOLIS, MN, 55440-1022, US

(21) Appl. No.: 11/207,181

(22) Filed: Aug. 19, 2005

(51) Int. Cl. A01N 1/02; C12Q 1/68; G01N 33/53; C12M 3/00

(52) U.S. Cl. 435/2; 435/6; 435/7.1; 435/287.2

(57) ABSTRACT

A biological specimen is preserved by surrounding the biological specimen with a protective enclosing structure. The protective enclosing structure reacts with corrosive components in the surrounding atmosphere or environment to reduce their degradative effect and also protects the preparations from bacterial and fungal growth.

### TECHNICAL FIELD

This description relates to protecting and preserving biological specimens. In particular, this description relates to protecting and preserving biological specimens from the effects of exposure to corrosive chemicals and contaminants.

### BACKGROUND

Biological specimens include cytology specimens, biomolecule specimens, or cellular specimens.

Cellular specimens are routinely used in clinical medicine and biomedical research to determine gene and protein expression levels in disease lesions. Typically, cellular samples are processed into microscope slide preparations for subsequent testing and analysis. Common microscope slide preparation methods include cutting 2-14 micron sections with a microtome from cellular samples embedded in suitable embedding medium, such as paraffin or frozen sectioning embedding medium. Other cellular preparations include cytospins, smear preparations, nuclear touch-preparations and other similar samples containing tissues, cells or cell fragments.

Such cellular preparations can be stored for a variable amount of time before processing with appropriate analytical techniques. In clinical laboratories that use the preparations for diagnostic purposes, the preparations are typically stored only for a short time ranging from hours to days before analysis. However, in research use, the storage time can be considerably longer, ranging from days to several years.

Examples of well-known analysis methods include in situ techniques such as, for example, immunohistochemistry and nucleic acid hybridization. Cellular preparations attached onto a microscope slide are also commonly used to extract nucleic acids, proteins and other biomolecules from defined cellular regions for subsequent solution-based assays. A variety of microdissection techniques are known. Examples of microdissection methods include manual mechanical dissection with needles. Laser beams have been used to catapult regions of tissue into extraction solutions, or used to activate an adhesive tape that lifts region of tissue.

In situ and solution-based assays performed on cellular preparations or on molecules extracted from such preparations depend on preserving biomolecules in a condition that is as similar as possible to the condition at the time of sampling. However, biomolecules in stored cellular preparations are known to be unstable. Exposure to air is known to be a major factor affecting biomolecule stability. For example, archived slide preparations are known to show weaker immunoreactivity than freshly cut slide preparations from the same archived tissue source, demonstrating that biomolecules in embedded tissue are more stable than in microscope slide preparations. Analysis results can therefore be affected depending on how long the slide preparations were stored before assays are performed. To ensure unbiased results, slide preparations would have to be processed while fresh, or stored only for a short period of time prior to processing with immunohistochemistry, in situ hybridization or microdissection. The requirement for fresh preparations has several practical disadvantages that generally limit research laboratory throughput and increase assay costs. For example, histotechnicians have to retrieve tissue or cell blocks from storage archives, which is time consuming and laborious. The blocks need to be leveled before good quality new sections can be obtained, and this process consumes tissue and limits the number of assays that can be performed from each tissue block. This aspect is particularly disadvantageous with highly valuable multi-specimen blocks, such as tissue microarray blocks. In addition, some specimens, such as smear preparations, exist only in a microscope slide format and cannot be prepared fresh for research use.

Several methods have been described for protecting biological material in cellular preparations. However, these methods all have limitations. For example, microscope slides have been dipped into molten paraffin to provide protective coating, but this has been shown to be insufficient (Jacobs, T. W. et al., Loss Of Tumor Marker-Immunostaining Intensity On Stored Paraffin Slides Of Breast Cancer, J Natl Cancer Inst (1996) 88, 1054-1059). In addition, coating slides with paraffin is messy and clumsy. Storing cut slides at about 4° C. or −20° C. has also been suggested. This method is more effective than paraffin-coating, but may still be insufficient to prevent the loss of immunoreactivity (van den Broek, L. J., and van de Vijver, M. J. Assessment Of Problems In Diagnostic And Research Immunohistochemistry Associated With Epitope Instability In Stored Paraffin Sections, Appl Immunohistochem Mol Morphol (2000) 8, 316-321; Wester, K., et al., Paraffin Section Storage And Immunohistochemistry: Effects Of Time, Temperature, Fixation, And Retrieval Protocol With Emphasis On P53 Protein And MIB1 Antigen, Appl Immunohistochem Mol Morphol (2000) 8, 61-70). In addition, storage in cooled space is associated with high costs and consumption of limited laboratory space. Another method for protecting nucleic acids from degradation involves sealing the isolated nucleic acids into a cavity containing inert gas, such as nitrogen (U.S. Pat. No. 6,258,320; DiVito, K. A., et al, Long-Term Preservation of Antigenicity on Tissue Microarrays, Lab. Investigation (2004) 84, 1071-1078). This method affords good protection from degradation but requires complicated processing because the specimen must be sealed into a storage container. This approach is particularly cumbersome when access to the stored preparations is needed at multiple time intervals, as is common in research.

Other methods have been proposed for protecting materials such as metals, comic books, coins, and film from corrosion or degradation due to contact with atmospheric gases or fungal growth. These methods involve enclosing the material to be protected in a reactive polymer enclosure.

### SUMMARY

In one general aspect, biological specimens are surrounded with a protective enclosing structure formed, at least in part, from a barrier layer. Components of the ambient atmosphere or environment surrounding the enclosing structure permeate into and through the barrier layer, which reacts with these components and reduces or eliminates their degradative effect on the enclosed biological specimens.

In another general aspect, a method for protecting a biological specimen from degradation includes obtaining a container that includes a polymer including a transition metal, and placing a biological specimen in the container such that degradation of the biological specimen is inhibited.

In another general aspect, a container protects biomolecules in cellular preparations from degradation. The container is formed from a material that includes a polymer, where the polymer includes a transition metal. The container is capable of protecting a biomolecule from degradation.

In another general aspect, a method of conducting an assay on a biological specimen includes obtaining a biological specimen and performing an assay on the biological specimen, wherein activity of the biological specimen has been preserved. The biological specimen is obtained from a storage container that includes a reactive polymer that includes a transition metal.

In addition, in another aspect, an apparatus includes a means for holding a slide preparation in a container and a means for reducing the permeation of chemicals or contaminants or both through the container.

In a further general aspect, a container preserves a biological specimen and inhibits degradation of the biological specimen. The container includes a reactive polymer that includes a transition metal, and the container includes a holder for maintaining the biological specimen in a fixed position inside the container.

In another aspect, a method for protecting biological specimens from degradation includes enclosing a biological specimen together with a transition metal containing polymer in a container having low permeability to components of the ambient atmosphere.

In another aspect, the container can be a room, such as a hermetically sealed room, incorporating the reactive polymer. The reactive polymer can be in the form of a barrier layer that at least partially covers the room walls, floors and ceiling. Alternatively, the reactive polymer can be incorporated into an air recirculation system or a filtration system through which air in the room is passed to remove contaminants.

The method and apparatus provide an easy, cost-effective way for protecting biological specimen from degradation.

Other features will be apparent from the description, the drawings, and the claims.

### DESCRIPTION OF DRAWINGS

FIG. 1 is a perspective view of a container according to one implementation.

FIG. 2 is a plan view of a base of a container according to another implementation.

Like reference symbols in the various drawings may indicate like elements.

### DETAILED DESCRIPTION

This description generally relates to protecting biological specimen from degradation. A biological specimen can be one or more of a cytology specimen, a biomolecule specimen, or a cellular specimen. Degradation may occur as a result of the biological specimen's exposure to chemical substances or contaminants. These chemical substances and contaminants can be components of the ambient atmosphere surrounding the biological specimen. Degradation involves deterioration of biomolecules within the biological specimen and can result in their elemental breakdown and loss of immunoreactivity. The deterioration can adversely affect the accuracy and validity of assays, immunohistochemistry analyses, and in situ hybridization and microdissection procedures performed on the biological specimen.

Examples of cytology specimens include: gynecologic smears such as Pap smears; sputum samples; brushings such as bronchial, gastric, or esophageal brushing; washing such as bronchial or gastric washings; fluids such as urine, cerebral spinal fluid, pleural fluid, or abdominal fluid; synovial fluid; fine needle aspiration material; tumor touch samples; and seminal fluid. To form a Pap smear, cells from the cervix or vagina are removed and then examined for cancer to abnormal hormonal conditions. A fine needle aspiration is a minimally invasive method of obtaining cells for biopsy from any area of the body. Sputum samples are mucus or other materials produced by the lining of the respiratory tract, and are sometimes referred to as phlegm, though can include mucus, blood, and pus. Brushing, washing, and fluid samples are collected from various organ sites and used for detection of abnormal cells, malignant cells, and infectious agents.

Examples of biomolecule specimens include: metaphase chromosome spreads; protein arrays, and DNA arrays. Metaphase cells are used to prepare a standard karyotype, and chromosome spreads are produced from a population of dividing cells, such as lymphocytes. Metaphase spreads are used for cytogenetic and molecular cytogenetic tests, including assays such as fluorescence, in situ hybridization, comparative genetic hybridization, multicolor karyotyping, and spectral karyotyping. Protein arrays are measurement devices used in biomedical applications to determine the presence and/or amount of proteins in biological samples. Usually a multitude of different capture agents, most frequently monoclonal antibodies, are deposited on a solid surface in a miniature array. DNA arrays are arrays of DNA molecules bound to a solid surface that facilitates high throughput analysis of thousands of genes simultaneously. Bound DNA molecule size often varies depending on whether the array will be used for determining nucleotide polymorphisms, gene expression levels, or gene copy number evaluations. DNA arrays are used for probing a biological sample to determine gene expression, a marker pattern, or a nucleotide sequence of DNA/RNA.

Biomolecule specimens can also be made up of artificial molecules that are fragments, analogues, or variants of naturally occurring molecules. Artificial biomolecules may be synthesized by modifying naturally occurring biomolecules or by creating molecules from artificial ingredients and reagents. Artificial biomolecules can include, for example, cDNA arrays, artificial RNA molecules, and artificial proteins and antibodies.

Examples of cellular specimens include: conventional histology slides; cell line control slides; cell line arrays; tissue microarrays; and transfected cell microarrays. Transfected cell microarrays are slides prepared by culturing mammalian cells on a glass slide printed in defined locations with solutions containing different DNS's. Cells growing on the printed areas take up the DNA, creating spots of localized transfection within non-transfected cells. These arrays are used for multiparallel functional analysis of mammalian cells.

Cellular specimens may be in the form of microscope slide preparations containing biomolecules, or in the form of cytospins, smear preparations, nuclear touch-preparations and other similar samples containing tissues, cells or cell fragments. Alternatively, cellular specimens can be in the form of tissue arrays as described, for example, in U.S. Pat. No. 6,136,592 and U.S. Pat. No. 6,103,518 to Leighton.

Degradation of a biological specimen is reduced or prevented by surrounding the biological specimen with a protective enclosing structure formed, at least in part, from a barrier layer. Components of the ambient atmosphere or environment surrounding the enclosing structure permeate into and through the barrier layer, which reacts with these components and reduces or eliminates their degradative effect on the enclosed biological specimen.

The technique for reducing or preventing biological specimen degradation can be advantageously applied to any biological specimen, including cellular specimens that are routinely used in clinical medicine and biomedical research, cytology specimens, and biomolecule specimens. In general, biological specimens can contain biomolecules or biological materials such as DNA molecules, RNA molecules, protein molecules, cells, tissues, or organisms that have been removed from environments where they occur in nature. Techniques for removing biological specimens from their natural environment include, for example, excision, extraction, purification, slide preparation, array preparation, or collection of an animal, such as, for example, an insect.

The barrier layer can be semi-permeable or permeable with respect to the surrounding atmosphere or environment and can include multiple components or layers arranged in various configurations. For example, a permeable barrier layer can be in the form of a porous material, made up of a solid matrix with interconnected pores dispersed with in the solid matrix interstices, which allows the surrounding atmosphere to flow through the pores of the material. In such materials, components in the permeating fluid contact and react with the material at the pore walls. Some permeation into the dense matrix material may also occur, allowing for internal reactions within the matrix. On the other hand, in semi-permeable barrier layers, significant permeation occurs through the dense barrier layer material, which contains few pores through which fluids can flow. Permeation of certain components is favored over others and reactions in these semi-permeable membranes largely occur within the matrix material.

Barrier layers can be formed from a single material or from multiple materials. As described above, in barrier layers containing multiple materials, the materials can be arranged into a laminate or a layered structure. Alternatively, a first material may form a continuous or discontinuous phase dispersed within the interstices of at least a second barrier layer material.

Examples of reactive polymers are compounds in which reactive materials are incorporated into a polymer matrix. In one implementation, the reactive materials are solid-state. More specifically, such reactive polymers are polymers incorporating a corrosive gas reactant material such as a transition metal, which is optionally catalyzed to become part of the polymeric structure. Such reactive polymers were developed by AT&T Bell Laboratories (currently known as Lucent Bell Labs Technologies), and are disclosed in U.S. Pat. No. 4,944,916 issued to Franey. Similarly, they are discussed in John Franey, A New Permanent ESD and Corrosion Resistant Material, EOS/ESD Symposium Proceedings 1991, EOS/ESD Association, Rome N.Y. (which association has a website at www-esda.org), and are discussed at www-staticintercept.com. The reactive polymers act to neutralize corrosive gases commonly associated with corrosion and tarnishing, preventing the gases from interacting with a protected biological specimen.

Transition metals are generally suitable for use in the reactive polymers, and copper and aluminum are preferred in some implementations. Copper is a transition metal that has been known to be a passive mildewcide and fungicide when in intimate contact with an object.

The composition of suitable reactive polymers can cover a broad range with respect to the quantity of transition metal it contains. The weight percentage of transition metal in a polymer can be selected by striking a balance between maximum reactive polymer activity and adequate polymer strength. The presence of a transition metal in some polymers can cause a physical degradation of polymer properties (U.S. Pat. No. 4,944,916). However, generally such polymer degradation does not unacceptably reduce the strength of the polymer until a weight percentage of dispersed metal reaches substantially greater than 35 weight percent. See supra. Moreover, in some cases, the protection of biological specimens can be increased by an increase in the weight percent of transition metal in the polymer. As such, the maximum weight percent of transition metal is used to provide acceptable polymer strength.

In some implementations, the strength of the reactive polymer may be of little importance. In such cases, a wider range of weight percentage of dispersed metal may be suitable. For example, according to one aspect, a method for protecting biological specimens from degradation involves enclosing a biological specimen together with a quantity of reactive polymer in a container having low permeability to components of the ambient atmosphere. The permeability of such containers is preferably similar to the permeability exhibited by containers made from substantially dense or non-porous polymer or metal. Suitable container materials can include, for example, polypropylene, ABS or polyvinyl chloride, low-density polyethylene, and thermosetting polymer such as a polyester thermoset material. A sufficient quantity of the reactive polymer should be placed in the container with the biological specimen and shaped to present a sufficient surface area to absorb and react with contaminants present in the container at a sufficient rate to adequately reduce degradation of the biological specimen.

The introduction of a protecting metal into a polymer can be accomplished by any suitable method. By way of example, the introduction of a protecting metal into a polymer may be accomplished by one of several conventional polymer chemistry processing techniques. For example, the metal in the form of particles can be introduced into a polymer by internal mixers as described in “Polyethylene”, R. A. V. Raff and J. B. Allison, Interscience Pub., Ltd., New York, p. 399 (1956). The polymer can be formed into pellets by the “Caviar Cut” method as described in Raff and Allison, and then formed into the desired structures by procedures such as extrusion blow molding (described in DuPont Magazine (1949)).

The transition metal can be concentrated in a particular area, such as, for example, on the polymer surface or in a discrete layer within the polymer, or alternatively, the transition metal can be dispersed throughout the polymer. In one implementation, the transition metal is dispersed throughout the polymer. A metal dispersed throughout a polymer can be dispersed randomly or in any other arrangement. In another implementation, the polymer contains a substantial surface area of one or more transition metals.

A wide variety of polymers is suitable. Hydrocarbon-based polymers are particularly suitable. For example, a polymer matrix such as polypropylene, ABS or polyvinyl chloride can be used. As another example, polymers that are easily formed into bags such as low-density polyethylene can be used. Thermosetting materials that are formed into structurally supported plates are also employable. For example, in this manner, a polyester thermoset material can be formed into a box. The reactive polymers can be those available from Engineered Materials, Inc. of Buffalo Grove, Ill. under the trade names Static Intercept.^{RTM} and Corrosion Intercept.™

Reactive polymers can be manufactured by catalyzing copper material into polymer chains to form a homogeneous polymeric/metallic structure of low-density polyethylene (LDPE). Optionally, manufacture of reactive polymers can further involve the additional catalysis of C12 into the poly-metallic structure to form a copper/metal/oxide semi-conductive media, which utilize the “Bucky Ball” phenomena to provide paths for electron flow within the structure. The LDPE structures are formed into standard pellets and processed into various final structures, the most common of which is blown film to manufacture bags.

When a biological specimen is enclosed within a reactive polymer container, degradation from the environment trapped within the enclosing structure is significantly less than the corrosion induced by permeation of components of the ambient air surrounding the container. In some environments, these components can include, for example, reactive oxygen, gaseous sulfur and chlorine-containing entities. Moreover, the permeation of corrosive materials is substantially reduced by the presence within the polymer of a scavenger such as, without limitation, copper or aluminum. For example, a container can be produced by introducing copper or aluminum particles into a polymer and then forming the polymer, for example, low density polyethylene, into a container. Additionally, even in relatively severe environments, protection against degradation can be maintained for extended periods of time. Optionally, to avoid contaminating the protected biological specimen, the barrier layer can be fabricated from non-volatile, or minimally volatile substances. This reduces the likelihood of barrier layer materials vaporizing from the barrier layer and subsequently condensing on and contaminating the biomolecules.

An enclosing structure can be fabricated using the barrier layers generally described above. The enclosing structure is a container that includes a barrier layer configured so that a significant proportion of corrosive or degradative components in the surrounding atmosphere or environment pass through the barrier layer to enter the enclosing structure. However, as described above, the barrier layer protects enclosed biological specimens by reacting and/or reducing the degradative effect that the permeating components may have on the biological specimen. For example, a container can include a reactive polymer that includes a transition metal, in which the container inhibits degradation of the biological specimen; and a holder for maintaining the biological specimen in a fixed position inside the container.

A container can include a device for holding a biological specimen. For example, a container includes without limitation a bag, a box, a bottle, a jar, a canister, a tube, a room, or any other apparatus for holding one or more biological specimens. A container can be any shape, including for example, spherical, oval, cubic, pyramidal, or any other shape, including an irregular shape. A container can be of any degree of rigidity, including for example rigid, flexible, malleable, stretchable, or soft. The surfaces of the container can have any pH. For example, the surfaces of the packaging preferably have a chemically neutral pH (in the approximate range of 7.0 to 7.5).

A container can be made entirely from a reactive polymer. Alternatively, a container can be coated or lined with a reactive polymer or a product containing the reactive polymer. For example, a container can be constructed from paper or foam core materials as described in U.S. Pat. No. 6,593,007 (Donaldson). The paper-based materials can be made of cellulose-based materials, which are of high alpha-cellulose content and are negative to lignin side chains. In this non-limiting example, the surfaces of paper or foam core products are treated for the purpose of inhibiting degradation of a biological specimen, and the paper or foam core is formed into a container. In another example, a container is lined or coated with a paper or foam core product such as that described in Donaldson.

Optionally, containers can be designed so that, when closed, they have reactive polymer on the inside surfaces. In addition, adhesives are preferably avoided in the container. Alternatively, adhesives that are employed do not possess chemicals that contribute to the degradation of the biological specimen.

A container can be any thickness sufficient to inhibit degradation of the biological specimen. A container may have any dimensions sufficient to hold one or more biological specimens. A container has at least one opening through which a biological specimen can be placed in the container and removed from the container as necessary. An opening can be any structure by which access may be gained to the inside of the container. The opening can be sealed to exclude the environment outside the container. The opening can be sealed by any means or combination of means, including, for example, a cap, a lid, a stopper, a door, a fold, an adhesive, a clip, an o-ring, or a screw. In one example, the opening can be hermetically sealed from the environment outside the container.

The container optionally can include more than one separate storage compartment for individual biological specimens. For example, the container includes partitions between separate storage compartments. As another example, the container optionally can block the deleterious effects of ultraviolet radiation on a biological specimen. The container can be transparent, translucent, or opaque. If the container is used to store a light-sensitive biological specimen, then the container can be opaque.

The container may incorporate desiccants to control interior humidity and maintain contained biological specimens at appropriate moisture levels. Desiccants can be included as a layer or film on the container walls, dispersed within the container's interior volume as loose particles or in the form of an insert, such as a slide-shaped insert. To facilitate rapid and reliable container identification, containers may also incorporate external labeling, such as radio frequency identification (RFID) chips or bar codes encoded with an identifier for the container. These RFID chips and bar codes can be read using sensing devices and bar code readers that are known in the art and can be used to identify the contents of the container without unnecessarily exposing the biological specimens to contaminants.

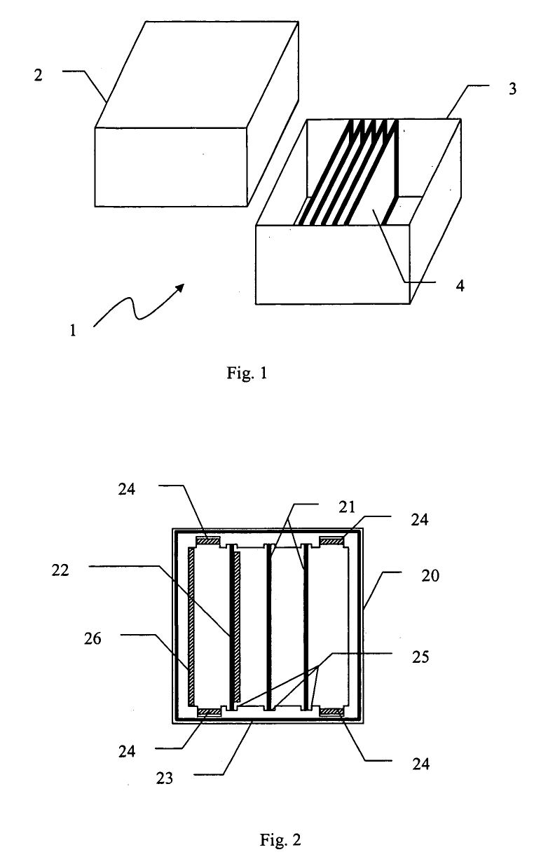

Referring to FIG. 1, the container is a box 1 having a base 3 and a lid 2. The box 1 includes one or more slides 4 on which are deposited biological specimens. The sides of the box 1 can incorporate a reactive polymer in the form of a reactive polymer coating on the interior surfaces of the box 1, for example. The container optionally includes one or more portals (not shown) through which the air inside the container can be replaced with an inert gas, such as for example, argon.

In one implementation, the container includes, or is formed in the shape of a holder. The holder can be any mechanism that holds, cradles, fastens, or restrains a biological specimen. For example, without limitation, a holder can be a receptacle for a tissue array, a notch or a recess in the inner surface of the container for a slide, a slide box that the container comprises, a Velcro strip, a screw, or a clip.

Referring to FIG. 2, in another implementation, container is a box-shaped container that has a base 20. The base 20 includes notches 25 for securely and removably holding biological specimens on slides 21. The notches 25 can also hold inserts or slides incorporating the reactive polymer 22 or other materials, such as desiccants, that may be useful for preserving the biological specimens. Also, as discussed earlier, container walls may incorporate reactive polymer on inner surfaces of the container, such as on the walls of the base 20, which can be coated with a film 26 of reactive polymer. The container can also include other receptacles 24 for holding reactive polymer or desiccants. Optionally, the container can be closed by forming an air-tight or an hermetic seal using the lid 2 (as shown in FIG. 1) or some other form of closure. For example, to facilitate an air-tight or hermetic seal, the base 20 can incorporate an elastomeric gasket or seal 23 against which an appropriate lid could close to prevent the influx of air from outside the container, as illustrated in FIG. 2.

A method for protecting biological specimens from degradation includes obtaining an enclosing structure having a barrier layer, placing the biological specimens within the enclosing structure so that the barrier layer reduces the degradative effect of components of the surrounding atmosphere or environment that permeates into or through the barrier layer. A substantial proportion of the surrounding atmosphere that enters the enclosing structure permeates through the barrier material. For example, more than about 25% of the surrounding atmosphere permeates through the barrier. As another example, more than 50% of the surrounding atmosphere permeates through the barrier. As a further example, more than 75% of the surrounding atmosphere permeates through the barrier.

A method for protecting biological specimens further includes providing a container that includes a polymer that includes a transition metal, and placing a biological specimen in the container such that degradation of the biological specimen is to some extent inhibited. Optionally, these methods for protecting biological specimens from degradation can further include flushing or otherwise replacing the atmosphere within the enclosing structure with an inert gas and sealing the enclosing structure so that contaminants present in the enclosing structure are removed.

In one aspect, the enclosing structures and the methods described above can be used to preserve a biological specimen, that is, to protect a biological specimen from degradation. In another aspect, the enclosing structures and the methods described above can be used to protect a biological specimen from degradation by inhibiting the rate of degradation of the biological specimen by at least about 25%, at least about 50%, at least about 70%, at least about 80%, or at least about 90%.

The biological specimen is protected from degradation by reducing, inhibiting, or preventing destruction of the biological material of the specimen. Any means can be used to determine whether degradation of a biological specimen has been reduced, inhibited, or prevented. For example, analysis of a control sample can be made from all or a portion of a test sample prior to placing the test sample in a container, and the test sample can then be subjected to conditions that tend to degrade the biomolecules of the specimen. For example, conditions that degrade biomolecules that can be used on the test sample are exposure to one or more of: nucleases, solutions of chlorine bleach, hydrogen sulfide, ozone, and ultraviolet light. After exposure, the test sample can be retrieved from the container and analyzed for integrity. The amount of degradation can be assessed by comparing the results of analysis of a control sample with the results of the same analysis on the test sample.

The enclosing structures and the methods described above can be used to preserve or protect a biological specimen from degradation by reducing, inhibiting, or preventing the loss of antigenicity of the preparation. Alternatively, the enclosing structures and the methods described above can be used to protect a biological specimen from degradation by maintaining the stability of an epitope in a tissue section. For example, protecting a biological specimen from degradation can involve preserving the sample to the degree that histochemical results upon assay of the biological specimen are unaltered, minimally altered, or only slightly altered when compared to the results at the time of sample preparation. Where assay results, including immunostaining results, are unaltered, no difference is detected between assay of the biological specimen at the time of storage and assay after storage for a period of time. Minimal alteration of histochemical results refers to a difference of less than about 20% between assay of the biological specimen at the time of storage and assay after storage for a period of time. Slight alteration of histochemical results refers to a difference of less than about 50% between assay of the biological specimen at the time of storage and assay after storage for a period of time.

Any suitable method can be used to verify that the biological specimen is maintained in a condition substantially the same as the condition of the biological specimen when the preparation was made. For example, according to one technique where the preparation is a second slide containing biomolecules, immunostaining of the second slide may be compared with immunostaining of a first control slide that was prepared prior to placing the second slide in the container (See Jacobs, 1996). In an alternative technique, where the biological specimen is DNA, maintenance of the DNA can be verified by gel electrophoresis, restriction endonuclease analysis, DNA sequencing, or any other suitable technique or combination of techniques.

The enclosing structures and the methods described above can also be used for improving cellular assays by storing cellular preparations used in the assays in the enclosing structures described above prior to their use in assays. For example, a method of improving an assay on a sample of a biological specimen can include obtaining a sample of a biological specimen that has been stored in a reactive polymer container, which polymer includes a transition metal; and performing an assay on the sample of the biological specimen, where the activity of the sample has been preserved. An assay on a sample of a biological specimen can include performing an analysis, test or evaluation or combination thereof on all or a portion of the biological specimen. This can involve obtaining a sample of a biological specimen that has been stored in a container. A sample of a biological specimen can include all or any portion of the biological specimen.

Performing an assay can include, for example, immunostaining for any antigen, such as immunostaining for p53 in breast cancer cells. See Jacobs et al., “Loss of Tumor-Marker Immunostaining Intensity on Stored Paraffin Slides of Breast Cancer”, Journal of the National Cancer Institute, 88(15): 1054-1059 (1996).

Other implementations are within the scope of the following claims.

What is claimed is:

1. A method for protecting a biological specimen from degradation comprising:
providing a protective enclosing structure having a barrier layer; and
placing the biological specimen in the protective enclosing structure, such that degradation of the biological specimen is inhibited.

2. The method of claim 1, wherein the biological specimen is a slide preparation.

3. The method of claim 2, wherein the protective enclosing structure is a container having a microscope slide holder.

4. The method of claim 1, wherein the biological specimen is located on a tissue array.

5. The method of claim 4, wherein the protective enclosing structure is a container having tissue array holder.

6. The method of claim 1, wherein the barrier layer includes a reactive polymer.

7. The method of claim 6, wherein the reactive polymer includes a transition metal.

8. The method of claim 7, wherein the transition metal is copper or aluminum.

9. The method of claim 1, further comprising introducing an inert gas into the protective enclosing structure.

10. The method of claim 1, wherein the biological specimen comprises a protein array.

11. The method of claim 1, wherein the biological specimen comprises a DNA array.

12. The method of claim 1, wherein the protective enclosing structure contains a desiccant.

13. The method of claim 1, further comprising labeling the protective enclosing structure with an RFID tag or a bar code.

14. A method of conducting an assay on a biological specimen comprising:
obtaining a biological specimen stored in a protective enclosing structure; and
performing an assay on the biological specimen, wherein the activity of the biological specimen is substantially preserved.

15. The method of claim 14, wherein the protective enclosing structure is a container having a tissue array holder.

16. The method of claim 14, wherein the protective enclosing structure is a container having a microscope slide holder.

17. The method of claim 14, wherein the protective enclosing structure is a container that comprises a reactive polymer.

18. The method of claim 17, wherein the reactive polymer includes a transition metal.

19. The method of claim 18, wherein the transition metal is copper or aluminum or both.

20. The method of claim 14, wherein the protective enclosing structure further comprises a desiccant.

21. The method of claim 14, wherein the biological specimen is a cytology specimen.

22. An apparatus comprising:
a protective enclosing structure;
a means for a holding a cellular preparation in the protective enclosing structure; and
a means for reducing the permeation of chemicals or contaminants or both through the protective enclosing structure.

23. The apparatus of claim 22, wherein the means for reducing the permeation of chemicals or contaminants or both includes a reactive polymer.

24. The container of claim 22, wherein the protective enclosing structure includes a label that identifies a biological sample within the protective enclosing structure.

25. The container of claim 24, wherein the label includes an RFID tag or a bar code.

26. A container for preserving a biological specimen, the container comprising:
a reactive polymer having a transition metal dispersed therein; and
a holder for maintaining a tissue array or a microscope slide preparation in a fixed position inside the container.

27. The container of claim 26, wherein the transition metal is copper or aluminum.
